(19) United States
(12) Patent Application Publication
(10) Pub. No.: US 2007/0280906 A1
(43) Pub. Date: Dec. 6, 2007

(54) Method to generate mirrored adenoassociated viral vectors

(76) Inventor: Ognjen Petras, San Francisco, CA (US)

Correspondence Address: Ognjen Petras, Apt. #8, 1350 Sutter Street, San Francisco, CA, 94109, US

(21) Appl. No.: 11/422,078

(22) Filed: Jun. 3, 2006

(51) Int. Cl. A61K 48/00; C12N 15/861; C12N 5/06

(52) U.S. Cl. 424/93.2; 435/456; 977/802; 435/325; 435/457

(57) ABSTRACT

The present invention describes mirrored adenoassociated virus genomes that can spontaneously fold to form double-stranded DNA structures capable of directing efficient RNA transcription in mammalian cell nuclei. Also described are mirrored adenoassociated viral particles that incorporate the mirrored vector genome and a suitable adenoassociated viral capsid. Further described are DNA templates and methods for producing the mirrored adenoassociated vector genomes and mirrored adenoassociated viral particles. Methods of administering these reagents to mammals are also described as are specific in vitro and in vivo applications where the mirrored adenoassociated virus has unique utility.

### RELATED APPLICATION INFORMATION

This application claims the benefit of U.S. Provisional Application No. 60/208,604 which was filed Jun. 3, 2005, This application described the present invention (mirrored adenoassociated viruses and viral genomes) using a different name: “functionally double-stranded DNA vectors”. These so named “functionally double-stranded DNA vectors are identical to the mirrored adenoassociated virus vectors described in the preceding claims of the present patent application. Furthermore the provisional application described the same method for vector synthesis detailed in the present patent application.

### FIELD OF THE INVENTION

The present invention relates to reagents for gene therapy. In particular the present invention relates to improved adenoassociated virus-based gene delivery vectors.

### LANGUAGE, TERMS, AND REFERENCES

The following sections will describe the background of the invention and discuss in greater detail the features of the viral genomes and methods disclosed in the claims section. We will use scientific terms commonly used by molecular biologists and virologist that are easily understood by those skilled in these arts. Abbreviations are in italicized, bold text and their first instance immediately follows their full spelling. References to publications are listed as (Ref 1), (Ref 2), etcetera. A bibliography for these references can be found at the end of the document. References to figures are listed as (FIG. 1), (FIG. 2), etcetera.

### BACKGROUND OF THE INVENTION

In recent years, recombinant viral vectors based on the adenoassociated virus (AAV) have emerged as promising vehicles for gene therapy in a variety of contexts. Their useful traits include an ability to mediate long-term gene expression in most mammalian organs, and a good safety profile in early human clinical trials.

Individual AAV particles consist of a non-enveloped protein capsid that encases one single-stranded DNA (ssDNA) genome. Recombinant AAV genomes are devoid of wild-type viral sequences except for two 145 nucleotide inverted terminal repeat sequences (ITRs) located at the 5′ and 3′ genome termini. The ITR encodes nucleotide sequences that are required for genome replication and virus particle assembly in virus producing cells. AAV-ITR sequences can vary between individual wild-type AAV isolates (AAV serotypes). However almost all recombinant AAVs in current use contain AAV serotype-2 specific ITR sequences. With the aid of a synthetic strategy known as pseudo-typing a recombinant AAV genome with AAV serotype-2 specific ITR sequences can be packaged with many different AAV capsid serotypes. An important limitation of the AAV genome is its limited information carrying capacity. AAV genomes have a maximum size limit of about 5,000 nucleotides of sequence. Genomes above this size can still be replicated and be packaged in virus producing cells. However oversized genomes package in a defective fashion and generate viral particles with reduced or absent infectivity.

First generation AAVs used in recent and ongoing human clinical trials are designed around semi-partite genomes. This means that each individual first-generation AAV genome encodes either the sense or anti-sense strand of a double-stranded DNA (dsDNA) sequence. Vectors of this sort (referred to below single-stranded AAVs or ssAAVs), provided safe, durable and therapeutic gene expression in several preclinical studies such as Factor IX replacement in mice and dogs. However other preclinical studies revealed that the semi-partite nature of ssAAV genomes severely limits their gene delivery capabilities.

In general dsDNA templates are essential components of transcription, the process by which polypeptide sequences encoded on individual DNA strands are converted into messenger RNA molecules. Consequently semi-partite ssAAV genomes must be provided with a complementary ssDNA strand in order to direct efficient expression of a gene. Complementary DNA strands can be synthesized in an infected cell by using the ssAAV genome as a template for second-strand synthesis, Alternatively, complementation can be provided by annealing of individual, complementary ssAAV genomes within an infected cell. Unfortunately complementation is an inefficient process. In many cell types a phospho-regulated nuclear protein, FK506BP-55kD, adheres to a specific portion of the AAV-ITR, and prevents second strand DNA synthesis or annealing of individual complementary ssAAV genomes. Furthermore uncomplemented ssAAV genomes are unstable species in cell nuclei and undergo brisk degradation unless they convert into dsDNA molecules.

Maneuvers such as transient adenoviral gene expression can release FK506BP-55kd binding at the ITR and improve AAV mediated gene expression by factors of 10-100 in vitro. Similarly FK506BP-55kd inactivation in transgenic mouse livers enhances AAV mediated hepatocyte transduction by a factor of 20. For obvious technical reasons these maneuvers could be exceedingly difficult to accomplish in humans patients. Hence the full clinical potential of first generation AAVs is limited by the ssAAV genome. A germane example of this limitation is the poor hepatocyte transduction efficiency of ssAAVs in adult mice:

In adult murine livers massive vector doses (fifty trillion viral genomes per kg of body weight) of ssAAVs with AAV serotype-2 capsids can transduce less than 12% of hepatocytes (Nakai et al, J Virol. November 2002; 76(22):11343-9). This limitation can be overcome (in mice) by ssAAVs with novel, recently isolated AAV capsid types such as serotype-8 at vector doses of fifty trillion vector genomes per kg of body weight. However this amount of virus would be impractical and extremely expensive to produce for a human-sized subject. In non-human primates ssAAVs with a variety of capsid serotypes including the extremely efficient serotype-8 also have limited hepatocyte transduction efficiencies at practical vector doses (one trillion viral genomes per kg of body weight) (Nathwani et al, Blood. Apr. 1, 2006; 107(7):2653-61).

We wanted to improve the transduction efficiencies of AAV vectors in hepatocytes in order to facilitate AAV based therapies for ornithine transcarbamylase deficiency (OTCD), an inborn error of hepatic metabolism. In previous studies recombinant replication defective adenoviral vectors, which allow efficient hepatocyte transduction, corrected OTCD in mice. Hence our chief technical goal was to generate AAVs with adenoviral-like hepatocyte transduction capabilities. In particular our goal was to introduce a functional gene into 30% or more of the hepatocytes of a mouse liver using an AAV vector.

To achieve this technical goal we generated novel mirrored adenoassociated viral genomes that fold into self-complementing dsDNA-like structures when released from their viral capsids (FIG. 1). This maneuver bypasses the genome conversion barrier that limits ssAAV efficiency. Mirrored adenoassociated viral genomes increased AAV mediated gene expression ten-fold or more in mouse livers when they were compared to a control ssAAV vector. In particular we reproducibly achieved hepatocyte transduction efficiencies greater than 50% using only three trillion viral genomes per kg of body weight. In general, mirrored adenoassociated viral vectors outperformed control ssAAVs in terms of relative gene expression by factors of ten or more in multiple contexts including in human hemopoetic stem cells.

### DETAILED DESCRIPTION OF THE INVENTION

In this section we will further discuss the claims listed in the preceding section of the patent application. We will also report data pertinent to the utility of the patent application and we will discuss some of the possible uses of the present invention. The specific examples offered below are not meant to limit the particular embodiments of the present invention. Rather, these examples are provided so that this disclosure will be thorough and complete, and will fully convey the scope of the invention to those skilled in the art.

### Claims 1 Through 11:

Claims one through eleven (1-11) describe the mirrored adenoassociated viral genome. This molecule is a key independent claim of the present patent application. Although the mirrored genome is physically a single-stranded DNA molecule, it has the capability to fold back on itself to form a double-stranded region of DNA. This capability allows AAV particles consisting of mirrored genomes to bypass the barriers of ssDNA to dsDNA genome conversion, which limit the gene expression efficiency of ssAAVs as was previously explained. As will be discussed claims 1-12 do not seek patent protection for the general concept of a ssDNA molecule that can form dsDNA structures by folding on itself since such a concept is rather obvious. Rather claims 1-11 seek patent protection for particular embodiments of foldable ssDNA molecules that are also AAV genomes. The specific structure of these foldable AAV genomes delimited by claims 1 through 11.

Claims 1-11 of the invention can be more thoroughly explained with respect to the specific identity of the nucleotides forming the “specific nucleotide domains” of the mirrored adenoassociated virus genome of claim 1. The two central complementary “recombinant nucleotide sequence” domains are arranged on a single strand of DNA in a head to head fashion without the presence of any intervening, non-complementary nucleotide sequences. By “recombinant” we signify that these sequences lack any substantial homology to wild-type AAV sequences and in particular to AAV-ITR sequences or other sequences that can provide AAV-ITR-like functions such AAV genome replication and packaging.

The lack of any intervening nucleotides distinguishes the mirrored adenoassociated virus genome from “packaged dimeric intermediates”. Dimeric intermediates are single-stranded DNA molecules that are generated during ssAAV genome replication and ssAAV virus production in virus producing cells. Dimeric intermediates are double-sized with respect to their derivate AAV genomes. They resemble mirrored adenoassociated virus genomes in that they can spontaneously fold to form regions of double-stranded DNA capable of directing efficient expression of a gene. However dimeric intermediates also have a third wild-type AAV-ITR sequence located in the middle of their genomes Dimeric intermediates below 5000 nucleotides in length are generated during the replication of ssAAV genomes less than 2500 nucleotides in length. In this setting the dimeric intermediates are similar in length to the wild-type AAV genome and are occasionally packaged as infectious adenoassociated virus particles. Unfortunately the vast majority of AAV viral particles prepared in this fashion will be ssAAV viral particles because most dimeric intermediates are cleaved at their central AAV-ITR sequence into ssAAV genomes before they can be packaged into a viral capsid. Nevertheless several published reports in the gene therapy literature have noted that ssAAV preparations containing minor amounts of packaged dimeric exhibit a several-enhanced gene delivery capability when compared to equal doses of ssAAV preparations lacking packaged dimeric intermediates. A comparison of mirrored adenoassociated virus genome structure and dimeric intermediate genome structure can be seen in FIG. 1.

The lack of a central AAV-ITR derived sequence also distinguishes mirrored adenoassociated viral genomes from the “duplexed parvovirus genome” described in U.S. patent Application No 20040029106 (Jude R Samulski et al). This patent describes adenoassociated viral genomes that also have the ability to fold into dsDNA-like structures. Like packaged dimeric intermediates duplexed parvovirus genomes also have a central AAV-ITR derived sequence. However the central AAV-ITR of duplexed parvovirus genomes is mutated. A functional nucleotide domain knows as the terminal resolution site is deleted from the central AAV-ITR. This mutation essentially traps the dimeric intermediate during AAV genome replication and allows the generation of “duplexed parvovirus”. Duplexed parvovirus preparations are largely composed of foldable AAV genomes that resemble dimeric intermediates. A comparison of mirrored adenoassociated genome structure and duplexed parvovirus genome structure can be seen in FIG. 1. A description of the duplexed parvovirus and the method of its synthesis was reported in the gene therapy literature by two independent research groups in simultaneous reports (Wang et al, Gene Ther. December 2003; 10(26):2105-1, and McCarty et al, Gene Ther. December 2003; 10(26):2112-8). Duplexed parvoviruses have also been named “self-complementary AAVs” or “double-stranded AAVs” by their various inventors.

Those educated in the biology and behavior of AAV vectors will appreciate that the perfectly symmetric central recombinant nucleotide sequences of mirrored adenoassociated virus genomes may provide certain advantages when compared with the genomes of packaged dimeric intermediates and duplexed parvoviruses.

Because of their central AAV-ITR sequences packaged dimeric intermediates and duplexed parvoviruses have a diminished capacity to encode useful recombinant nucleotide sequences. This is because of the relatively strict size limit of AAV genomes as explained in the background section. Because AAV vectors with foldable genomes (e.g. duplexed parvoviruses) encode both strands of a recombinant gene sequence their information carrying capacity is only one half that of ssAAV genomes. Based on a wild-type AAV genome size of 4700 ssDNA nucleotides and an AAV-ITR length of 145 nucleotides, this equals about 2220 nucleotides of dsDNA sequence for mirrored adenoassociated virus genomes and only 2070 dsDNA nucleotides for packaged dimeric intermediates or duplexed parvovirus genomes. Under these circumstances the added information capacity (about 6.8%) provided by mirrored adenoassociated virus genomes could be extremely useful especially in settings where one desires to equip a foldable AAV genome with a longer polypeptide encoding nucleotide sequence.

An additional advantage of the mirrored adenoassociated relates to an AAV related gene delivery strategy known as trans-splicing. Trans-splicing allows AAV vectors to deliver a gene that under normal circumstances would be too large to incorporate into a single AAV genome. The over-sized gene is divided into two fragments that are then packaged into separate AAV virus particles. The separate virus particles are then introduced into a cell. The separate genomes are released from their respective viral capsids and enter the nucleus. Subsequently the cell's DNA repair machinery recombines the separate genomes to regenerate the original over-sized gene. Since the reconstituted gene fragments are joined at the AAV genome ends, transcription of the reconstituted gene must occur across the joined genome ends. Since packaged dimeric intermediates and duplexed parvovirus genomes have AAV-ITR sequences at their genome termini trans-splicing strategies involving packaged dimeric intermediates or duplexed parvovirus genomes would require transcription across AAV-ITR sequences. Unfortunately AAV-ITR sequences are very GC rich (>90%) and form hairpin structures that can derail or block RNA transcription from a dsDNA template. Hence the unique ITR-less ends of mirrored adenoassociated viral vectors could be an aid to trans-splicing strategies since their genome ends can be designed to carry any desired sequence. This latter aspect of our technology will be further discussed in subsequent sections of this patent application.

Yet another advantage of the unique mirrored adenoassociated viral genome relates to a problem called “gene regeneration”. Partially defective AAV-ITR sequences such as those found in duplexed parvovirus genomes can be repaired during genome replication through recombination with wild AAV-ITRs. This could limit the ability of certain duplexed parvovirus preparations to be free of contaminating ssAAV genomes. By comparison mirrored adenoassociated viral genomes lack a mutated AAV-ITR sequence and are immune to the problem of gene rearrangement. From a different perspective, packaged dimeric intermediate and duplex parvoviruses are generated through a failure or subversion of the usual AAV genome replication machinery. Consequently the inherent error correction mechanisms of the AAV genome replication machinery thwart packaged dimeric intermediate and duplexed parvovirus synthesis. Conversely mirrored adenoassociated virus genomes employ the normal AAV genome synthesis program and benefit from its error correction mechanisms.

Those skilled in the art of AAV vector design will appreciate that recombinant AAV virus particles can be produced from templates incorporating a variety of serotype-specific AAV-ITR sequences if virus producing cells are also provided with the corresponding serotype-specific AAV Rep gene. Hence claim 2 discloses that any AAV vector genome equipped with a pair of functional AAV-ITRs at the 5′ and 3′ genome ends and possessing a central pair of complementary “recombinant nucleotide sequences” oriented in a head-to-head fashion constitutes a mirrored adenoassociated viral genome. By “functional AAV-ITRs” we mean that these sequences can direct replication and packaging of AAV genomes in virus producing cells. From a different perspective, the specific geometrically symmetric arrangement of central the recombinant sequence as specified in claims 1,6, and 7 is the core novel innovation of the current invention. By contrast the AAV-ITR regions of the viral genome represent a different field of AAV vector design and innovation.

Those skilled in the art of AAV vector design will also appreciate that engineered sequences not found in wild-type AAV isolates can provide functions comparable to bona-fida AAV-ITR sequences. For example 5′ portions of the AAV Rep gene can direct replication and packaging of recombinant nucleotide sequences in the absence of an AAV-ITR. Hence claim 3 further expands the classes of AAV vector genomes claimed by the current patent application to include any AAV vector genome possessing a central pair of complementary “recombinant nucleotide sequences” as delimited by claims 1,6 and 7 where the genome has non AAV-ITR genome ends that can nevertheless provide AAV-ITR related functions.

Those skilled in the art of AAV vector design will appreciate that AAV vector genomes can be encapsidated into a nearly endless variety of AAV capsid types some of which have been isolated from nature and some of which have been extensively engineered. The mirrored adenoassociated viral genome increases gene expression independently of any advantages offered by a particular AAV capsid type. Hence claim 10 emphasizes that any AAV viral particle with a mirrored adenoassociated viral genome should be considered a mirrored adenoassociated viral particle regardless of the capsid type employed in the particular embodiment of the viral particle.

Those skilled in the art of gene therapy vector design will appreciate that the utility of viral gene therapy vectors stems from their ability to deliver nucleotide sequences to cell nuclei. Nucleotide sequences can than mediate a variety of useful effects on the cell depending of their functional domains. Claims 8 lists some commonly used functional nucleotide domains whereas claim 9 lists several peptide sequences that can be or have been incorporated into mirrored adenoassociated viral genomes by the inventors. They (claims 8 and 9) disclose that any AAV genome whose central recombinant nucleotide domain is structured according to claims 1,6,7 shall be considered a mirrored adenoassociated virus genome regardless of the specific identity or utility of the “recombinant nucleotide sequences” of claims 1,2,3,6,7,8, and 9.

### Claims 12 Through 43

Claims 12-26 describe linear dsDNA templates that can direct replication and packaging of mirrored adenoassociated viral genomes in permissive cells. The unusual template used to generate mirrored adenoassociated viral genomes and particles distinguishes them from ssAAV vectors and the duplexed parvovirus vectors. In the case of ssAAV vectors the dsDNA template employed consists in the 5′ to 3′ direction of: (i) A first wild-type AAV-ITR sequence; (ii) A recombinant nucleotide sequence not derived from wild-type AAV sequences; and (iii) A second wild-type AAV-ITR sequence. In the case of duplexed parvovirus vectors the dsDNA template consists in the 5′ to 3′ direction of: (i) A first wild-type AAV-ITR sequence; (ii) A recombinant nucleotide sequence not derived from wild-type AAV sequences; and (iii) A second mutated AAV-ITR sequence. By comparison the templates used for mirrored adenoassociated virus synthesis require only a single AAV-ITR sequence. Furthermore, it will be appreciated by those with a knowledge of AAV vector synthesis that the templates used to generate ssAAVs and dimeric parvoviruses can be delivered to permissive cells as either linear dsDNA molecules or circular dsDNA molecules without affecting the success of the AAV synthetic process, By comparison the synthesis of mirrored adenoassociated virus genomes and particles requires a linear dsDNA template to succeed. We will disclose experimental data supporting this claim in subsequent sections of this patent application. FIG. 2 depicts the linear template of claim 12.

An important aspect of the template of claim 12 is the orientation of the D or “terminal resolution site” domain of the single AAV-ITR sequence with respect to the two ends of the linear dsDNA molecule. Recombinant nucleotide sequences located between a free dsDNA end and the D sequence of the AAV-ITR are incorporated into AAV capsids when the linear template is delivered to virus producing cells. In contrast, the sequence located between the other free dsDNA end and the other end of the AAV-ITR is not packaged as an AAV genome. FIG. 2 depicts the linear template of claim 12.

Those skilled in the art of AAV vector synthesis will appreciate that generating human scale and clinical grade qualities of AAV particles can be technically challenging and expensive. A particular requirement is the availability of decigram quantities of pure dsDNA templates such as the one specified claim 12. Claims 22-27 disclose an inexpensive methods to generate the linear dsDNA templates of claim 12: A circular dsDNA molecule with sequences identical to the linear substrate of claim 12 is propagated in a micro-organism, harvested, and purified. The circular molecule encodes a strategically placed restriction endonuclease recognition sequence that is digested with the same restriction endonuclease to generate the linear template of claim 12. In a preferred embodiment of this method the “recombinant nucleotide sequence that is not incorporated into the mirrored adenoassociated virus genome” of claims 12 and 27 consists of a bacterial antibiotic resistance gene and a high-copy bacterial origin of replication. The specific sequence of the strategically placed endonuclease recognition sequence is chosen so that inexpensive commercially available restriction endonucleases can be used to convert the circular template of claim 27 into the linear template of claim 12. The digested DNA can then be purified by any suitable method including organic solvent extraction and affinity column chromatography. A useful and flexible aspect of this method is that any restriction endonuclease that cuts the circular template of claim 27 at only one location can be employed to generate the linear template of claim 12. FIG. 2 depicts the circular template of claim 27.

### Claims 44 Through 52

As previously explained dimeric intermediates are ssDNA molecules generated during the synthesis of AAV genomes. Since they are derivatives of the linear template of claim 12 and are precursors to the mirrored adenoassociated viral genome of claim 1 the present patent application claims the specific dimeric intermediate delimited in claims 44-52. Experimental data proving the existence of the dimeric intermediates of claims 44-52 will be disclosed in subsequent sections of this patent application.

### Claims 53 Through 65

These claims related to a method to produce mirrored adenoassociated virus particles using the templates of claim 12 and 44. Adenoassociated viral vectors are typically generated by transfecting cultured mammalian cells permissive to virus production with a set of dsDNA molecules that direct assembly of virus particles. One group of dsDNA molecules encodes the adenoassociated and adenoviral helper genes necessary to direct replication and packaging of AAV genomes as viral particles. These helper genes are usually supplied to the cells as one or two circular dsDNA molecules. Alternatively these helper genes can be delivered with the aid of viral vectors. Furthermore, helper genes can be supplied by virus-producing cells if the cells'genomes contain copies of the requisite helper genes. Whereas helper genes allow cells to make AAV particles templates such as the templates of claims 12 and 44 determine which specific nucleotide sequences are incorporated into the genomes of AAV particles as they are assembled. Numerous specific AAV synthesis methods based on the above schemes have been patented and are available to researchers. Therefore the current patent application does not seek to restrict the rights to use these methods except where the templates of claims 12 and 44 are used to generate the mirrored adenoassociated virus genomes and particles of claim 1.

### Claims 65 Through 75

Claims 65-75 describes the delivery of mirrored adenoassociated viral genomes and particles to cells or subjects. It will be appreciated that many specific techniques and routes can be used to deliver AAV genomes and particles to cells or subjects. Therefore the current patent application does not claim exclusive the rights to use these methods except where the mirrored adenoassociated virus genomes and particles of claim 1 are the gene therapy reagent being delivered. Similarly a myriad of possible applications exist for gene therapy reagents some of which are listed in claims 65-75. Therefore the current patent application does not claim exclusive rights to use AAV vectors for these applications except where the mirrored adenoassociated virus genomes and particles of claim 1 are the gene therapy reagent being delivered.

### Data Relevant to the Present Patent Application

The presence of a foldable nucleotide region in AAV genomes generated by the method of claim 58 was confirmed by denaturing agarose gel electrophoresis and by the ability of genomes purified from viral particles to be digested with restriction endonucleases that only cut dsDNA molecules.

The presence of the dimeric templates of claim 44 was confirmed by restriction digestion and southern blotting of DNA extracted from virus producing cells that were transfected with the template of claim 12. Furthermore when we transfected virus producing cells with the circular templates of claim 27 they failed to generate the dimeric intermediates of claim 44. Hence linearization of the circular templates described in claim 27 appears to be critical component of the method of mirrored adenoassociated virus production method disclosed in the current patent application.

Several dsDNA templates conforming to the specifications of the templates of claims 12 and 27 were used to generate pure high-titer preparations of mirrored adenoassociated viral particles. For example a circular dsDNA molecule conforming to the template of claim 27 was constructed. This molecule, named, pFR-CMV-RFP contained several unique restriction enzyme sites including BsrGI, SpeI, HincII, and EcoRI. We digested pFR-CMV-RFP with BsrGI, SpeI, HincII, or EcoRI and transfected the linearized template into HEK293 cells along with the helper plasmids necessary for serotype-2 encapsidated AAV production. Transfection of AAV packaging HEK293 cells with BsrGI or EcoRI digested pFR-CMV-RFP produced discretely sized AAV genomes that differed in molecular weight. Based on their estimated molecular weight and the size of their restriction digest fragments, we concluded that sequences between the BsrGI, SpeI, HincII, or EcoRI restriction site and the flanking AAV-ITR of pFR-CMV-RFP had been faithfully replicated and packaged as mirrored adenoassociated virus genomes. (FIG. 3).

It will be appreciated from the above example that an advantage of the template of claim 27 and of the method of claim 58 is that the same circular template can be used to produce mirrored AAV genomes incorporating different regions of a circular template. This accomplished by cutting the circular template of claim 27 at different locations with different restriction enzymes. Although our experimentation was not exhaustive we currently believe that any restriction endonuclease site can be used in the circular template of claim 27 without affecting the success of the method of claim 58.

The chief utility of mirrored adenoassociated viral genomes is that they greatly enhance the efficiency of AAV mediated gene in vitro and in vivo.

In cultured mammalian cells mirrored adenoassociated viruses were compared to ssAAV genomes in terms of relative gene expression for matched virus doses. In HEK293 cells serotype-2 encapsidated fluorescent protein reporting mirrored adenoassociated virus genomes enhanced gene expression by factors of 20 or more on a genome per genome basis. We then tested mirrored adenoassociated viruses in non-transformed cells. In cultured non-dividing adult rat hepatocytes fluorescent protein reporting mirrored adenoassociated viruses outperformed ssAAVs by factors of 10-15 and reproducibly achieved transduction efficiencies of 30% or greater at high multiplicities of infection (ten thousand virus particles per cell). We then tested the ability of the mirrored adenoassociated virus to mediate long-term gene expression in a population of normal dividing cells. Human hemopoetic stem cells were infected with graded doses of a GFP reporting mirrored adenoassociated virus or a control ssAAV preparation. Gene expression was quantified by flow cytometry two weeks after infection. During this time period the dividing stem cells underwent a 500-1000 fold or greater increase in population numbers. Mirrored adenoassociated viral genomes reproducibly provided 10-20 times as much long-term transduction when compared to ssAAVs genomes. At the highest doses mirrored adenoassociated viruses with serotype-1 capsids allowed stable long-term gene expression in up to 9% of the stem cells.

It will be appreciated by those with a knowledge of AAV vector biology that AAV vectors mediate long-term gene expression in dividing hemopoetic stem cells by recombining with stem cell chromosomal DNA. Hence an additional advantage of mirrored adenoassociated viruses is that they have enhanced integration capabilities in the setting of cell division when compared to ssAAV vectors.

As stated above, our chief technical motivation for developing the present invention was to enhance AAV mediated gene delivery in the liver. To test mirrored adenoassociated viruses in the liver adult male mice received portal vein injections of three hundred billion vector genomes of GFP reporting ssAAVs or mirrored adenoassociated viruses. These vectors were synthesized with AAV serotype-6 capsids. In murine livers serotype-6 encapsidated AAVs reach peak gene expression levels within two weeks of administration. Two weeks post-injection livers receiving mirrored adenoassociated viruses achieved an average hepatocyte transduction efficiency of 37.2% (range 16%-66%, n=5). Control ssAAV injected livers attained an average transduction efficiency of 2.1% (range 0.9%-3.3%, n=4). The difference was statistically significant (p=0.02 for an unpaired t-test).

It will be appreciated by those with a knowledge of human liver diseases that inborn errors of metabolism often target the liver and could be cured by efficient gene delivery to liver cells. Therefore it will be appreciated that a chief potential use of mirrored adenoassociated vectors would be to treat diseases such as ornithine transcarbamylase deficiency. Additionally the liver secretes many vital blood proteins including coagulation proteins such as Factor IX. Therefore a second chief use of mirrored adenoassociated virus vectors would be to increase the amount of secreted gene products that could be produced by cells such as hepatocytes that were treated with an AAV vector.

From a different perspective mirrored adenoassociated viruses are advantageous because they can provide gene expression equivalent to that of a ssAAV at much lower doses of viral particles. Although AAVs are exceptionally safe with regard to generating immune responses, immune responses to components of the viral capsid have been reported in human and animal subjects. Hence mirrored adenoassociated viruses could deliver the same gene expression as ssAAVs with a lower risk of an immune response because subjects receiving gene therapy would be exposed to lower amounts of viral capsid proteins.

### Text for Figures

FIG. 1 depicts mirrored adenoassociated virus, packaged dimeric intermediate, and duplexed parvovirus genomes. The genomes are depicted as linear unfolded single stranded DNA molecules. The genome ends are labeled 5′ and 3′ according to convention. The functional nucleotide domains are labeled as follows: For mirrored adenoassociated virus genomes A to B denotes the first AAV-ITR sequence; B′ to B denotes the “terminal resolution site” domain of the first AAV-ITR; B to C denotes the first “recombinant nucleotide sequence”; C to D denotes the second “recombinant nucleotide sequence” which is essential the exact base-pairing complement of the first; D to E denotes the second AAV-ITR sequence; and D to D′ denotes the “terminal resolution site” domain of the second AAV-ITR. For packaged dimeric intermediates A to B denotes the first AAV-ITR sequence; B′ to B denotes the “terminal resolution site” domain of the first AAV-ITR; B to C denotes the first “recombinant nucleotide sequence”; C to D denotes the second AAV-ITR; C to C′ and D′ to D denote the two “terminal resolution site” domains of the second AAV-ITR; D to E denotes the second “recombinant nucleotide sequence” which is essential the exact base-pairing complement of the first; E to F denotes the third AAV-ITR sequence; and E to E′ denotes the “terminal resolution site” domain of the third AAV-ITR. For duplexed parvovirus genomes A to B denotes the first AAV-ITR sequence; B′ to B denotes the “terminal resolution site” domain of the first AAV-ITR; B to C denotes the first “recombinant nucleotide sequence”; C to D denotes the second AAV-ITR which has been mutated to remove its “terminal resolution site” domain; D to E denotes the second “recombinant nucleotide sequence” which is essential the exact base-pairing complement of the first; E to F denotes the third AAV-ITR sequence; and E to E′ denotes the “terminal resolution site” domain of the third AAV-ITR. As can be seen the unfolded structures of the three types of AAV genomes differ substantially in structure. In particular mirrored adenoassociated virus genomes lack a central AAV-ITR domain. Because of this the folded mirrored adenoassociated virus genomes lack the hairpin structures formed by AAV-ITR sequences at one of their genome ends

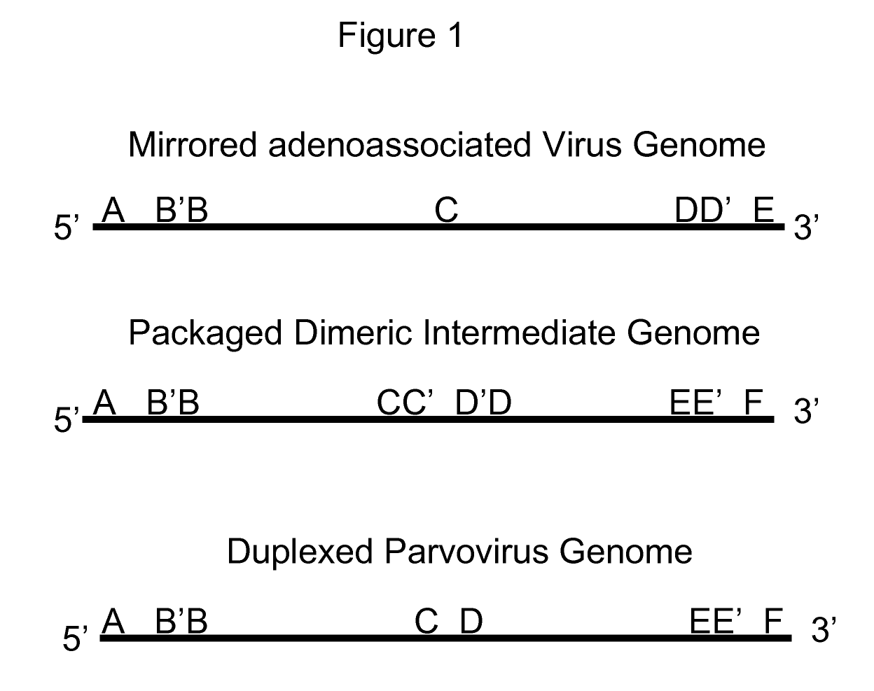

FIG. 2: Depicts the templates of claims 12 and 27. For the linear template A denotes a free linear dsDNA molecule end; A to B denotes the “the recombinant nucleotide sequence that is incorporated into the mirrored adenoassociated virus genome” of claim 12; B to C denotes the single AAV-ITR of the template; B to B′ denotes the “terminal resolution site” domain of the same AAV-ITR; C to D denotes “the recombinant nucleotide sequence that is not incorporated into the mirrored adenoassociated virus genome” of claim 12; and D denotes the other a free dsDNA molecule end of the template. A circular dsDNA molecule possessing a unique restriction endonuclease recognition sequence at site at A can be converted to the linear template by cutting the circular molecule with same restriction endonuclease.

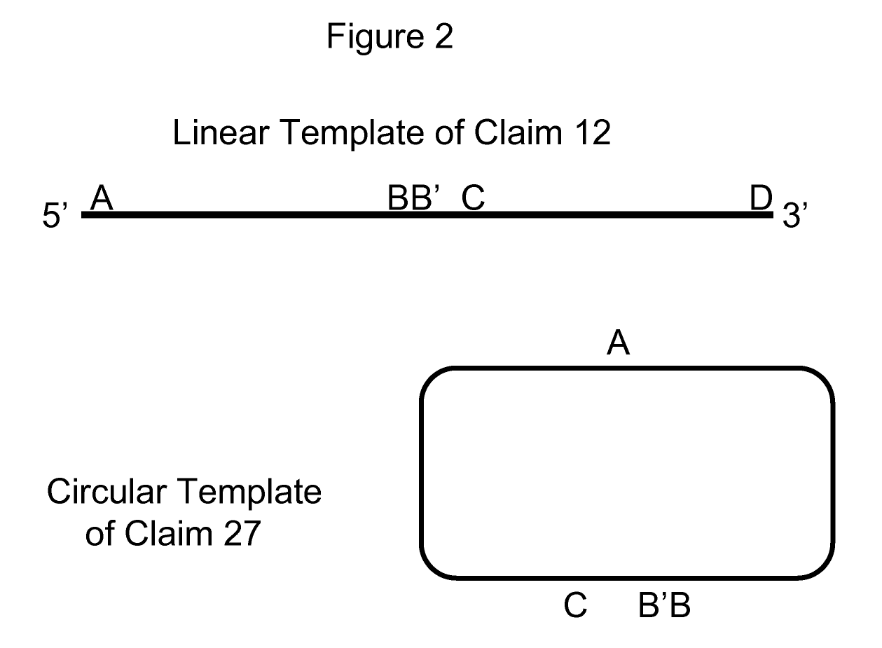

FIG. 3: Shows the experimental results described in a preceding paragraph.

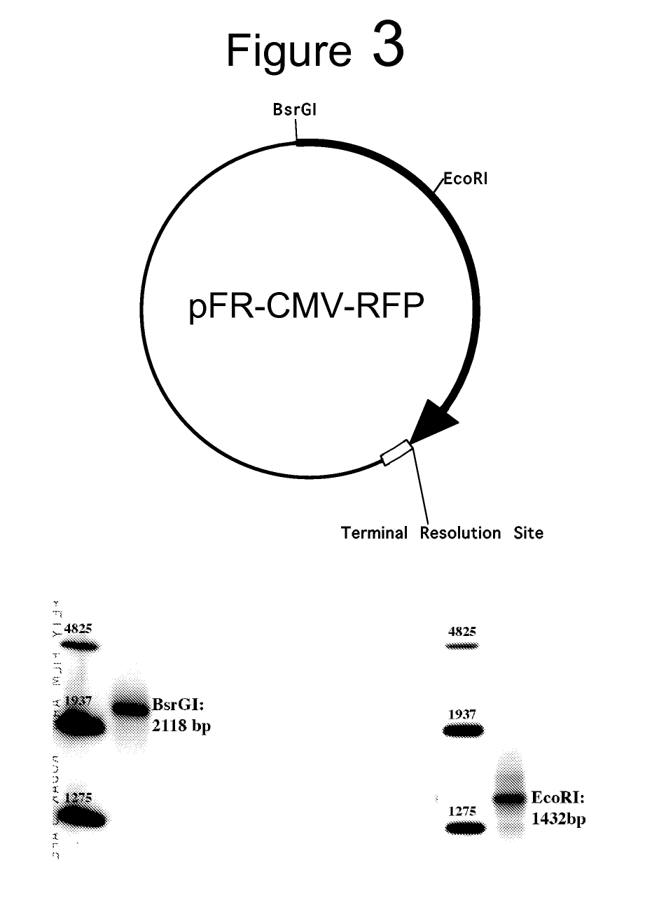

A circular template as described in claim 12 was digested with BsrGI or EcoRI. The template was transfected into AAV producing HEK293 cells. The thin black line represents the bacterial origin of replication and the bacterial antibiotic resistance gene. The thick black arrowhead represents the reporter gene. The short white region represnts the AAV-ITR. The terminal resolution site is labeled as such. The location of restriction endonuclease recognition sites is indicated on the diagram. The southern blot shows different sized bands representing different purified mirrored adenoassociated virus that were generated by linearizing the circular template at EcoRI or BsrGI and transfecting the linearized template into AAV packaging HEK293 cells.

1. A mirrored adenoassociated virus particle that is comprised of an adenoassociated viral: capsid and a mirrored adenoassociated virus genome. The vector genome is a single-stranded DNA molecule with the following specific nucleotide domains in the 5′ to 3′ direction: (i) A 5′ adenoassociated virus terminal repeat sequence; (ii) A first recombinant nucleotide sequence followed immediately by a second recombinant nucleotide sequence that is essentially the exact base-pairing complement of the first recombinant nucleotide sequence; and (iii) A 3′ adenoassociated virus terminal repeat sequence. Since the annealing of complementary single-stranded DNA molecules is an exothermic process under physiological conditions, mirrored adenoassociated virus genomes spontaneously fold into double-stranded DNA-like structures when they are liberated from their viral capsids in the intra-cellular environment.

2. The mirrored adenoassociated virus genome of claim 1 wherein said adenoassociated viral terminal repeat sequences are selected from any naturally occurring adenoassociated virus strains including AAV1, AAV2, AAV3, AAV4, AAV5, AAV6, AAV7, AAV8, and AAV9.

3. The mirrored adenoassociated virus genome of claim 1 wherein said adenoassociated viral terminal repeat have been replaced with naturally derived or engineered DNA sequences that can direct packaging and replication of adenoassociated viral genomes.

4. The mirrored adenoassociated virus genome of claim 1 wherein the length of the genome is equivalent to approximately 50-100% of the wild-type AAV genome length. This equals approximately 2400-4800 nucleotides of sequence.

5. The mirrored adenoassociated virus genome of claim 1 wherein said adenoassociated viral terminal repeat sequences have the following orientation of with respect to the “D” and the “terminal resolution site” sequences located within the same terminal repeats: The “D ” and “terminal resolution site” sequences are located 3′ to the ends the viral genome and are 5′ to the complementary recombinant nucleotide sequences.

6. The mirrored adenoassociated virus genome of claim 1 wherein the first and second “recombinant nucleotide sequences” are devoid of adenoassociated viral terminal repeat sequences derived from any naturally occurring adenoassociated virus strains including AAV1, AAV2, AAV3, AAV4, AAV5, AAV6, AAV7, AAV8, and AAV9.

7. The mirrored adenoassociated virus genome of claim 1 wherein the first and second “recombinant nucleotide sequences” are devoid of naturally derived or engineered DNA sequences that can direct packaging and replication of adenoassociated viral genomes.

8. The mirrored adenoassociated virus genome of claim 1 wherein the first and second “recombinant nucleotide sequences” encode any, some, or all of the following functional domains: (i) Promoter sequences that direct RNA transcription from a double-stranded DNA template; (ii) Enhancer sequences that increase RNA transcription from a promoter sequence; (iii) Silencer sequences that can block transcription from promoter sequences when placed in physical proximity to a promoter sequence on the same DNA molecule; (iv) Nucleotide sequences that encode a polypeptide sequence (i.e. a protein); (v) Polyadenylation sequences that direct efficient polyA tailing of nascent messenger RNA molecules; (vi) Nucleotide sequences capable of generating short-hairpin RNA molecules which silence cell gene expression via the cellular RNA interference system; and (vii) Intronic sequences which can stabilize messenger RNAs generated by a viral vector.

9. The mirrored adenoassociated virus genome of claim 8 wherein the polypeptide sequence is selected from a group including: human ornithine transcarbamylase, human Rb1, human plasminogen, human inhibitor of nuclear-factor kappa-B (IKB), and human TRAIL.

10. The mirrored adenoassociated virus particle of claim 1 wherein said adenoassociated viral capsid is selected from any naturally derived or engineered capsid serotype including but not limited to AAV1, AAV2, AAV3, AAV4, AAV5, AAV6, AAV7, AAV8, or AAV9.

11. A pharmaceutical formulation comprising a plurality of the mirrored adenoassociated virus particles of claim 1 in a suitable pharmaceutical delivery vehicle.

12. A sequence of specific nucleotide domains comprising a template for producing the mirrored adenoassociated viral genomes and mirrored adenoassociated viral particles of claim 1. The template sequence consists of a linear double-stranded DNA molecule with the following specific nucleotide domains in the 5′ to 3′ direction: (i) A 5′ free double stranded DNA end; (ii) A recombinant DNA sequence that is incorporated into the mirrored adenoassociated virus genome; (iii) An adenoassociated virus inverted terminal repeat; (iv) A nucleotide sequence that is not incorporated into the mirrored adenoassociated virus genome; and (v) A 3′ free double-stranded DNA end.

13. The template of claim 12, wherein said adenoassociated viral terminal repeat sequences are selected from any naturally occurring adenoassociated virus strains including AAV1, AAV2, AAV3, AAV4, AAV5, AAV6, AAV7, AAV8, and AAV9.

14. The template of claim 12, wherein said adenoassociated viral terminal repeat have been replaced with naturally derived or engineered sequences that can direct packaging and replication of adenoassociated viral genomes.

15. The template of claim 12 wherein the combined length of the recombinant nucleotide sequence that is incorporated into the mirrored adenoassociated virus genome and the AAV inverted terminal repeat sequence is approximately 1200-2400 nucleotides of double-stranded DNA sequence.

16. The template of claim 12 wherein the length of the recombinant nucleotide sequence that is not incorporated into the mirrored adenoassociated virus genome is preferably 2500 nucleotides or greater in length.

17. The template of claim 12 wherein the D and “terminal resolution site” domains of the adenoassociated virus inverted terminal repeat are immediately 3′ to the first recombinant nucleotide sequence that is incorporated into the mirrored adenoassociated virus genome, are 5′ to the remainder of the inverted terminal repeat, and are 5′ to the second recombinant nucleotide sequence that is not incorporated into the mirrored adenoassociated virus genome.

18. The template of claim 12, wherein the two “recombinant nucleotide sequences” are devoid of adenoassociated viral terminal repeat sequences derived from any naturally occurring adenoassociated virus strains including AAV1, AAV2, AAV3, AAV4, AAV5, AAV6, AAV7, AAV8, and AAV9.

19. The template of claim 12, wherein the two “recombinant nucleotide sequences” are devoid of naturally derived or engineered DNA sequences that can direct packaging and replication of adenoassociated viral genomes.

20. The template of claim 12 wherein the “recombinant nucleotide sequence that is incorporated into the mirrored adenoassociated virus genome” encodes any, some, or all of the following functional domains: (i) Promoter sequences that direct RNA transcription from a double-stranded DNA template; (ii) Enhancer sequences that increase RNA transcription from a promoter sequence; (iii) Silencer sequences that can block transcription from promoter sequences when placed in physical proximity to a promoter sequence on the same DNA molecule; (iv) Nucleotide sequences that encode a polypeptide sequence (i.e. a protein); (v) Polyadenylation sequences that direct efficient polyA tailing of nascent messenger RNA molecules; (vi) Nucleotide sequences capable of generating short-hairpin RNA molecules which silence cell gene expression via the cellular RNA interference system; and (vii) Intronic sequences which can stabilize messenger RNAs generated by the viral vector.

21. The template of claim 20 wherein the polypeptide sequence is selected from a group including: human ornithine transcarbamylase, human Rb1, human plasminogen, human inhibitor of nuclear-factor kappa-B (IKB), and human TRAIL.

22. The template of claim 12 wherein the 5′ and 3′ free double-stranded DNA ends are blunt ended or contain 5′ or 3′ mono or polynucleotide overhangs.

23. The template of claim 12 wherein the recombinant nucleotide sequence that is not incorporated into the mirrored adenoassociated virus genome is preferably a nucleotide sequence that can direct retention and replication of circular double-stranded DNA molecules in microorganisms.

24. The template of claim 12 wherein the recombinant nucleotide sequence that is not incorporated into the mirrored adenoassociated virus genome is comprised preferably of a bacterial antibiotic resistance gene and an bacterial origin of replication sequence.

25. The template of claim 12 wherein the recombinant nucleotide sequence that is not incorporated into the mirrored adenoassociated virus genome preferably contains an E. coli antibiotic resistance gene and a high-copy E. Coli origin of replication.

26. The linear double-stranded DNA template of claim 12 wherein the linear molecule can be generated by digesting a precursor circular double-stranded DNA template with a single restriction endonuclease.

27. A circular double-stranded DNA template that can be used to generate the linear double-stranded DNA template of claim 12 comprised of: (i) a restriction endonuclease recognition sequence which occurs only once on the entire circular template; (ii) A recombinant DNA sequence that is incorporated into the mirrored adenoassociated virus genome; (iii) a adenoassociated virus inverted terminal repeat; and (iv) A nucleotide sequence that is not incorporated into the mirrored adenoassociated virus genome.

28. The circular double-stranded DNA template of claim 27 wherein said adenoassociated viral terminal repeat sequences are selected from any naturally occurring adenoassociated virus strains including AAV1, AAV2, AAV3, AAV4, AAV5, AAV6, AAV7, AAV8, and AAV9.

29. The circular double-stranded DNA template of claim 27 wherein said adenoassociated viral terminal repeat have been replaced with naturally derived or engineered DNA sequences that can direct packaging and replication of adenoassociated viral genomes.

30. The circular double-stranded DNA template of claim 27 wherein the combined length of the recombinant nucleotide sequence that is incorporated into the mirrored adenoassociated virus genome and the AAV inverted terminal repeat sequence is approximately 1200-2400 nucleotides of double-stranded DNA sequence.

31. The circular double-stranded DNA template of claim 25 wherein the length of the recombinant nucleotide sequence that is not incorporated into the mirrored adenoassociated virus genome is preferably 2500 nucleotides or greater in length.

32. The circular double-stranded DNA template of claim 27 wherein the D and “terminal resolution site” domains of the adenoassociated virus inverted terminal repeat are immediately 3′ to the first recombinant nucleotide sequence that is incorporated into the mirrored adenoassociated virus genome, are 5′ to the remainder of the inverted terminal repeat, and are 5′ to the second recombinant nucleotide sequence that is not incorporated into the mirrored adenoassociated virus genome.

33. The circular double-stranded DNA template of claim 27, wherein the two “recombinant nucleotide sequences ” are devoid of adenoassociated viral terminal repeat sequences derived from any naturally occurring adenoassociated virus strains including AAV1, AAV2, AAV3, AAV4, AAV5, AAV6, AAV7, AAV8, and AAV9.

34. The circular double-stranded DNA of claim 27, wherein the two “recombinant nucleotide sequences” are devoid naturally derived or engineered DNA sequences that can direct packaging and replication of adenoassociated viral genomes.

35. The circular double-stranded DNA template of claim 27, wherein the “recombinant nucleotide sequence that is incorporated into the mirrored adenoassociated virus genome.” encodes any, some, or all of the following functional domains: (i) Promoter sequences that direct RNA transcription from a double-stranded DNA template; (ii) Enhancer sequences that increase RNA transcription from a promoter sequence; (iii) Silencer sequences that can block transcription from promoter sequences when placed in physical proximity to a promoter sequence on the same DNA molecule; (iv) Nucleotide sequences that encode a polypeptide sequence (i.e. a protein); (v) Polyadenylation sequences that direct efficient polyA tailing of nascent messenger RNA molecules; (vi) Nucleotide sequences capable of generating short-hairpin RNA molecules which silence cell gene expression via the cellular RNA interference system; and (vii) Intronic sequences which can stabilize messenger RNAs generated by the viral vector.

36. The circular double-stranded DNA template of claim 27 wherein the polypeptide sequence is selected from a group including: human ornithine transcarbamylase, human Rb1, human plasminogen, human inhibitor of nuclear-factor kappa-B (IKB), and human TRAIL.

37. The circular double-stranded DNA template of claim 27 wherein the recombinant nucleotide sequence that is not incorporated into the mirrored adenoassociated virus genome is preferably a nucleotide sequence that can direct replication of circular double-stranded DNA molecules in microorganisms.

38. The circular double-stranded DNA template of claim 27 wherein the recombinant nucleotide sequence that is not incorporated into the mirrored adenoassociated virus genome is comprised preferably of a bacterial antibiotic resistance gene and a bacterial origin of replication sequence.

39. The circular double-stranded DNA template of claim 27 wherein the recombinant nucleotide sequence that is not incorporated into the mirrored adenoassociated virus genome preferably contains an E. coli antibiotic resistance gene and a high-copy E. Coli origin of replication such as Co1E1.

40. The circular double-stranded DNA template of claim 27 wherein the restriction endonuclease recognition sequence is located between the recombinant nucleotide sequence that is not incorporated into the mirrored adenoassociated virus genome and the recombinant nucleotide sequence that is incorporated into the mirrored adenoassociated virus genome.

41. The circular double-stranded DNA template of claim 27 wherein the restriction endonuclease recognition sequence is preferably recognized by commercially available and relatively inexpensive restriction endonucleases including EcoRI, PstI, BamHI, and BgIII, HincII, NheI, and NdeI.

42. The circular double-stranded DNA template of claim 27 wherein the restriction endonuclease recognition sequence when cut by the corresponding restriction enzyme generates blunt, free double-stranded DNA ends.

43. The circular double-stranded DNA template of claim 27 wherein the restriction endonuclease recognition sequence when cut by the corresponding restriction enzyme generates free double-stranded DNA ends with 5′ or 3′ nucleotide overhangs.

44. A dimeric template for producing mirrored adenoassociated viral genomes and mirrored adenoassociated viral particles. The dimeric template is a single-stranded DNA molecule with the following specific nucleotide domains in the 5′ to 3′ direction: (i) A 5′ wild-type adenoassociated virus terminal repeat sequence; (ii) A first recombinant nucleotide sequence followed immediately by a second recombinant nucleotide sequence that is essentially the exact base-pairing complement of the first recombinant nucleotide sequence; (iii) A second wild-type adenoassociated terminal repeat sequence; (iv) a third recombinant nucleotide sequence that is essentially identical to the first recombinant nucleotide sequence followed immediately by a fourth recombinant nucleotide sequence that is essentially identical to the second recombinant nucleotide sequence; and (v) a 3′ wild-type AAV inverted terminal repeat sequence.

45. The dimeric template of claim 44, wherein said adenoassociated viral terminal repeat sequences are selected from any naturally occurring adenoassociated virus strains including AAV1, AAV2, AAV3, AAV4, AAV5, AAV6, AAV7, AAV8, and AAV9.

46. The dimeric template of claim 44 wherein said adenoassociated viral terminal repeat have been replaced with naturally derived of engineered DNA sequences that can direct packaging and replication of adenoassociated viral genomes.

47. The dimeric template of claim 44 wherein the length of the genome is equivalent to 100-200% of the wild-type AAV genome length (this equals approximately 4800-9600 nucleotides of sequence).

48. The dimeric template of claim 44 wherein the D and “terminal resolution site” domains of the three, inverted terminal repeat sequences are immediately adjacent to the ends of the four “recombinant nucleotide sequences”.

49. The dimeric template of claim 44, wherein the “recombinant nucleotide sequences” are devoid of adenoassociated viral terminal repeat sequences derived from any naturally occurring adenoassociated virus strains including AAV1, AAV2, AAV3, AAV4, AAV5, AAV6, AAV7, AAV8, and AAV9.

50. The dimeric template of claim 44, wherein the “recombinant nucleotide sequences” are devoid naturally derived or engineered DNA sequences that can direct packaging and replication of adenoassociated viral genomes.

51. The dimeric template of claim 44, wherein the “recombinant nucleotide sequences” encode any, some, or all of the following functional domains: (i) Promoter sequences that direct RNA transcription from a double-stranded DNA template; (ii) Enhancer sequences that increase RNA transcription from a promoter sequence; (iii) Silencer sequences that can block transcription from promoter sequences when placed in physical proximity to a promoter sequence on the same DNA molecule; (iv) Nucleotide sequences that encode a polypeptide sequence (i.e. a protein); (v) Polyadenylation sequences that direct efficient polyA tailing of nascent messenger RNA molecules; (vi) Nucleotide sequences capable of generating short-hairpin RNA molecules which silence cell gene expression via the cellular RNA interference system; and (vii) Intronic sequences which can stabilize messenger RNAs generated by the viral vector.

52. The dimeric template of claim 44 wherein the polypeptide sequence is selected from a group including: human ornithine transcarbamylase, human Rb1, human plasminogen, human inhibitor of nuclear-factor kappa-B (IKB), and human TRAIL.

53. A mirrored adenoassociated viral genome produced from the dimeric template of claim 44 through cleavage of the same nucleotide sequence at its the terminal resolution site of its second, central inverted terminal repeat sequence.

54. A mirrored adenoassociated virus particle incorporating a viral genome generated by cleavage of the dimeric template of claim 44.

55. A cultured cell containing the mirrored adenoassociated viral genome of claim 1 where: (i) The viral genome is an episomal DNA molecule or (ii) Is a double-stranded DNA molecule that has recombined with the cells' genomic or mitochondrial DNA in a stable fashion.

56. A cultured cell containing the template of claim 12 where: (i) The viral genome is an episomal DNA molecule or (ii) Is a double-stranded DNA molecule that has recombined with the cell's genomic or mitochondrial DNA in a stable fashion.

57. A cultured cell containing the dimeric intermediate of claim 40 where: (i) The dimeric intermediate is an episomal DNA molecule or (ii) Is a double-stranded DNA molecule that has recombined with the cells' genomic or mitochondrial DNA in a stable fashion.

58. A method of producing the mirrored adenoassociated virus particle of claim 1. The method comprised of providing cells permissive for adenoassociated virus replication with: (i) A nucleotide sequence encoding a template according to claim 12 or claim 44, or the mirrored adenoassociated viral genome of claim 1; (ii) Nucleotide sequences sufficient to direct intracellular replication of a template or mirrored adenoassociated viral genome; (iii) Nucleotide sequences sufficient to package mirrored adenoassociated viral genomes into adenoassociated virus capsids such that replication and packaging of mirrored genomes into adenoassociated viral capsids efficiently generates mirrored adenoassociated viral particles comprising mirrored adenoassociated viral genomes encapsidated within adenoassociated viral capsids.

59. The method of claim 58 further comprising the step of collecting the mirrored adenoassociated virus particles.

60. The method of claim 58, further comprising the step of lysing the cell prior to collecting the mirrored adenoassociated virus particles.

61. The method of claim 58, wherein the adenoassociated rep and cap coding sequences are provided by a plasmid.

62. The method of claim 58, wherein the adenoassociated rep and cap coding sequences are stably integrated into the cell.

63. The method of claim 58, wherein the adenoassociated rep and cap coding sequences are delivered to the cell by viral vectors.

64. The method of claim 58, wherein the helper virus sequences necessary to produce adenoassociated virus particles are provided by a plasmid.

65. The method of claim 58, wherein the helper virus sequences necessary to produce adenoassociated virus particles are provided by infecting the cells with helper viruses including adenoviruses or herpesviruses.

66. A method of delivering a mirrored adenoassociated virus genome to a cell, comprising contacting a cell with the mirrored adenoassociated virus particle under conditions sufficient for the mirrored adenoassociated virus particle to enter the cell.

67. The method of claim 62, wherein the cell is selected from the group consisting of a cancer cells, tumor cells, central nervous system cells, peripheral nervous system cells, striated muscle cells, heart muscle cells, lung cells, liver cells, intestinal cells, neuroendocrine cells, vascular endothelial cells, retinal neurons, retinal pigmented epithelial cells, eye epithelial cells, mature blood cells, adult stem cells, embryonic stem cells, and fetal stem cells.

68. A method of administering the mirrored adenoassociated virus genome of claim 1 to a subject comprising administering the cell of claim 66 to the subject.

69. A method of administering a mirrored adenoassociated virus genome to a subject, comprising administering to a subject the mirrored adenoassociated virus particle of claim 1 in a pharmaceutically acceptable carrier.

70. The method of claim 69, wherein the subject is selected from a group of vertebrate animals consisting of fish, amphibians, reptiles, birds, and mammals.

71. The method of claim 69, wherein the subject is a mammal.

72. The method of claim 69, wherein the subject is a human subject.

73. The method of claim 69, wherein the subject is a cancer patient, a tumor patient, a patient with an inherited bleeding disorder, a patient with an immune system deficiency, a patient with an inborn error of metabolism, a patient with a degenerative retinal disease, a patient with a degenerative central nervous system disease, a patient with a degenerative peripheral nervous system, disease a patient with diabetes, a patient with hypertension, a patient with an auto-immune disorder, a patient with an infectious disease, a patient with a degenerative skeletal disease, a patient with a degenerative neuromuscular disease a patient requiring immunization against a pathogen, and a patient susceptible to cancer due to an inherited gene mutation.

74. The method of claim 69, wherein the mirrored adenoassociated virus particle is administered by a route selected from a group consisting of oral, rectal, transmucosal, transdermal, inhalation, intravenous, subcutaneous, intradermal, intracranial, intramuscular, intraarticular, intravitreal, intraperitoneal, intrathoracic, and subretinal.

75. The method of claim 69 wherein the mirrored adenoassociated virus particle is administered to a site selected from the group consisting of a tumor, the brain, the spinal cord, the heart, the lungs, a muscle, airway epithelium, the liver, the eye, and the pancreas.
